(19) United States
(12) Patent Application Publication
(10) Pub. No.: US 2014/0106300 A1
(43) Pub. Date: Apr. 17, 2014

(54) REFLECTIVE DENTAL APPARATUS

(71) Applicant: Giti Ghavamzadeh, Waban, MA (US)

(72) Inventor: Giti Ghavamzadeh, Waban, MA (US)

(21) Appl. No.: 13/649,195

(22) Filed: Oct. 11, 2012

(51) Int. Cl. A61C 3/00; A61B 1/32

(52) U.S. Cl. 433/140; 600/242; 433/31

(57) ABSTRACT

A tissue retractor, for use by dental practitioners, in order to provide clear viewage of a large sector of the oral cavity. The retractor includes a unibody construction in one embodiment, which is intended for attachment to any of the numerous standard dental instrument handles, with two oblong mirrors connected by a bridge, which is static in some embodiments and retractably dynamic in some embodiments. The retractor pulls oral tissue away from the field of work and allows a dental practitioner without the assistance of a Dental assistant to have easy access to the oral workspace and eliminates the need to continually maneuver mirrored tools during procedures.

### CROSS REFERENCE TO RELATED APPLICATION

This application claims the benefit of and takes priority from U.S. Application No. 61/620,096 filed on Apr. 4, 2012, the contents of which are hereby incorporated by reference.

### FIELD OF THE INVENTION

This invention relates to apparatuses and systems to promote ease and accuracy in dental procedures. More particularly, the instant invention relates to apparatuses and systems pertaining to dental tissue retraction, and more specifically to retractor attachments that allows the dental practitioner to have a clear oral workspace and allowing ease of placement and lack of a need to maneuver further oral instruments.

### BACKGROUND OF THE INVENTION

Dental instruments featuring mirrors or in combination, have long been used in the dental field to assist practitioners with viewing the teeth and gums from the various angles present in the oral cavity. These mirrors have historically been circular in shape and feature specialized handles for maneuverability particularly in the posterior part of the mouth.

Many of the concurrent circular mirrored systems have had to suffice for instances when work is performed on a small region of one tooth perhaps for viewing the tooth properly during a procedure. However, these concurrent systems require a Dental assistant to manually hold and place a second mirror or another retracting instrument so that the dental practitioner can then appropriately maneuver the instruments needed for performing the procedure inside the oral cavity.

Additionally, numerous prior art systems have featured double dental mirrors connected by a bridge piece. Some concurrent embodiments utilize a first mirror, a second mirror and a bridge section attached to a specialized handle that contains a longitudinal passage for carry in salvia or other debris out of the oral cavity. Said systems are dependent on the specialized handle for maneuvering and positioning purposes.

Although this genre of system and apparatus may be suited for the particular purpose for which it is addressed, the configurations provided by these systems are not suited for the purpose of dental tissue retraction, as hereinafter described. This stands particularly evident as these apparatuses are predicated on a multi angular view of the workspace however having to adjust the instrument and reposition it to obtain the view causes the procedure to take longer and ultimately leads to patient discomfort. Likewise a mirror requiring detailed positioning by nature requires a second person to assist the dental practitioner. Two people attempting to maneuver instruments in what can a very small oral cavity causes a more complicated procedure and again ultimately results in patient discomfort and a longer procedure time.

One of the greatest challenges a dental practitioner faces in operating in the oral cavity is space. The oral tissue of a patient can often obstruct the field and cause the practitioner to again need an assistant to push the tissue away by the use a separate tongue depressor or similar instrument.

No concurrent design has seen fit to combine a true tissue retractor with a mirrored instrument and in fact, many either are not adaptable to such designs or are intended to illustrate the lack of merit in the designs. Moreover, some concurrent design claim to pull tissue away from the workspace but due to its size and shape the functionality of that claim is limited. Another disadvantage of the prior art is that it requires adjustment and potential additional retraction devices which crowds the work area and can obstruct the dental practitioner's view.

Another great challenge faced by dental practitioners face surrounds treating patients in a timely manner. The longer that a patient is in a procedure the more costs go up and ultimately it leads to further discomfort for the patient. Although numerous concurrent systems have attempted to combine various dental instruments into one unit in order to eliminate the need for numerous tools and attributes, these systems focus on economy but still require the dentist to maneuver the instrument during the procedure and thus can still be time consuming to use.

Finally dental instruments generally tend to have a high cost and also, as there is a clear necessity for specialized tools, a practitioner is faced with a requisite amount of devices and therefore the need to store said tools and to be able to access these tools during a procedure. A goal for practitioners is to ensure a clean and organized preparation area in order for the procedure to be timely and to safeguard the patient's health. However, having to change tools and rewire or reconnect tools to power utilities, drains, or suction devices can not only be timely but risks further exposure to the patient and increases the chance of mistake or error.

Although many of the concurrent mechanisms possess multiple utilities, however this genre of apparatus normally features customized handle units requiring an additional plug in to dental machinery. Therefore, these qualify as yet another tool in entirety that the practitioner needs to store, lay out, and connect during a procedure.

Therefore an instrument, such as the instant apparatus, that can fully and easily retract tissue in larger volumes and does not require maneuvering during procedure and allows the practitioner to view the oral cavity clearly affords great utility. Finally a tool, such as the instant mechanism, that not only possesses and affords the user multiple utilities but can easily attach to and work with existing handle technologies to eliminate the need to excessive materials during a procedure.

### SUMMARY OF THE INVENTION

The instant apparatus and system, as illustrated herein, is clearly not anticipated, rendered obvious, or even present in any of the prior art mechanisms, either alone or in any combination thereof. The versatile system, method and series of apparatuses for creating and utilizing a dental tissue retraction and viewage system are illustrated. Thus the several embodiments of the instant apparatus are illustrated herein.

It is primary objective of the instant system to provide a dental practitioner with a clear quadrant of the oral cavity by retracting oral tissue and displaying lateral view of the working area. This attachment can easily be flipped or rotated and quickly positioned to any other quadrant of the oral cavity if needed. An additional goal of the present system and apparatus is to provide an instrument to be utilized by dental professionals to provide, for the user, a clear open well illuminated field of operation which allows performing dental procedures in every quadrant of the oral cavity, without risk of damaging the tongue, cheek, gingiva (gums) and lips of the patient.

It is yet another object of the present invention to provide an instrument that limits the amount of time a procedure takes due to its ease of use and capable functionality thus limiting patient discomfort. An additional goal of the present invention is to provide a reflective or mirrored oral tissue retractor that contains an oblong shape and is large enough in scale to create a clear workspace for an entire quadrant of the oral cavity.

It is yet another object of the present invention to provide a tissue retractor that contains at least two mirrors positioned laterally to allow a clear view of the working areas. It is yet another object of the present invention to provide a tissue retractor head that through a threaded shank, is capable of being attached to any common dental instrument handle.

It is yet another object of the present invention to provide a reflective oral tissue retractor to painlessly and on a larger scale provide retraction of oral tissue from the field of work in the oral cavity in order to provide lateral views of the working area using two opposing mirrors and to allow reflective light to illuminate the work area.

It is yet another object of the present invention to provide a reflective oral tissue retractor which can be used in all four quadrants of the mouth by flipping or repositioning the retractor and not having to change its structure.

It is an additional object of the present system to introduce a bridge section including a ratcheting or telescoping mechanism for retracting tissue and is capable of placement behind the backmost molar. It is also an objective of the instant system to introduce an apparatus to be used with any standard dental handle and a head that is easily attached to other handles using a threaded end and screw on motion.

To the accomplishment of the foregoing and related ends, certain illustrative aspects are described herein in connection with the following description and the annexed drawings. These aspects are indicative of the various ways in which the principles disclosed herein can be practice and all aspects and equivalents thereof are intended to be within the scope of the claimed subject matter. Other advantages and novel features will become apparent from the following detailed description when considered in conjunction with the drawings.

It is an additional object of the present system to introduce a bridge section including a ratcheting or telescoping mechanism for retracting tissue and is capable of placement behind the backmost molar. It is also an objective of the instant system to introduce an apparatus to be used with any standard dental handle and a head that is easily attached to other handles using a threaded end and screw on motion.

To the accomplishment of the foregoing and related ends, certain illustrative aspects are described herein in connection with the following description and the annexed drawings. These aspects are indicative of the various ways in which the principles disclosed herein can be practice and all aspects and equivalents thereof are intended to be within the scope of the claimed subject matter. Other advantages and novel features will become apparent from the following detailed description when considered in conjunction with the drawings.

The foregoing has outlined the more pertinent and important features of the present invention in order that the detailed description of the invention that follows may be better understood, and the present contributions to the art may be more fully appreciated. It is of course not possible to describe every conceivable combination of components and/or methodologies, but one of ordinary skill in the art may recognize that many further combinations or permutations are possible. Accordingly, the novel architecture described below is intended to embrace all such alterations, modifications, and variations that fall within the spirit and scope of the appended claims.

### BRIEF DESCRIPTION OF THE DRAWINGS

For a better understanding of the present invention, reference may be had to the following detailed description taken in conjunction with the accompanying drawings in which:

FIG. 1 illustrates isometric view of the instant tissue retractor, including a depiction of the attachment capability to a standard dental handle.

FIG. 2 illustrates a side view of the instant apparatus and further demonstrates the overall shape of the present apparatus and the uni-body structure

FIG. 3 illustrates an isometric view of the instant tissue retractor, emphasizing the overall shape and actual distance between the rectangular components and illustrating a standard dental handle attached to instant tissue retractor.

FIG. 4 illustrates an isometric view of the telescoping mechanism of the bridge arm.

FIG. 5 illustrates an isometric view of the ratcheting mechanism of the bridge arm.

FIG. 6 illustrates a front view of the mirrored surface of the retractor and its attachment to the unit's housing.

FIG. 7 illustrates an isometric view of an additional embodiment of an adjustable mechanism of within the bridge arm, according to the present invention.

FIG. 8 illustrates an isometric view of an additional embodiment of an adjustable mechanism of within the bridge arm and a bite block according to the present invention.

### DETAILED DESCRIPTION OF THE INVENTION

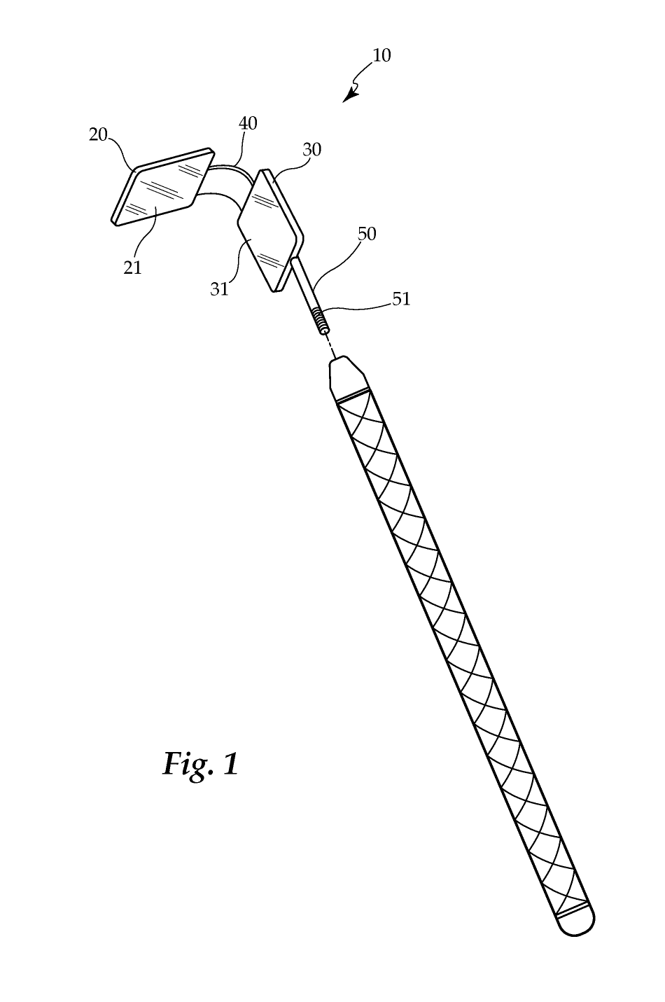

Turning now descriptively to the drawings, FIG. 1 illustrates the instant AyraDent™ tissue retractor system and apparatus 10 in use with a standard dental handle, wherein the retractor 10 is attached by the arm of the retractor 50 using a threaded end 51. In the instant embodiment, the AyraDent™ tissue retractor system comprises a uni-body attachment head 10 consisting of two substantially rectangular side pieces comprising substantially radial corners 20, that house two reflective surfaces, in one embodiment, mirrors 21, 31 which face each other and are connected by a bridge with a minimal curve 40. In numerous embodiments, mirrors 21, 31 are flat, concave, convex, multi surfaced or any combination thereof.

In one embodiment, the retractor 10 can come in two forms, the first being a disposable type preferably made of polycarbonate composite or other such material and constructed as a unitary housing the head and containing two front surface mirrors 21, 31. This form can also be made of autoclavable, inexpensive composite or another suitable plastic material. This composition allows for a uni-mold one piece assembly. The second form may be a metal-type constructed from stainless steel or a similar material that can be safely used in the human oral cavity and features a head composed of two rectangles with interior mirrors 21, 31 and a bridge 40 connecting the two rectangles. In the present invention the metal form includes a threaded end 51 on the arm 50 that extends from one rectangle 30 in order for the unit head to be attached to standard dental instrument handles. The instant apparatus allows the user to simply flip the retractor over for effective use on the left or right side of the patient's mouth.

The present apparatus can also be manufactured in a child size which would still allow for a full quadrant view of the oral cavity but can be proportioned to the standard size of a child. It is also possible that this retractor can be manufactured in various adult sizes to best suit the diversity of the size of adult oral cavities. Exact dimensions and materials used may vary depending on manufacturing needs.

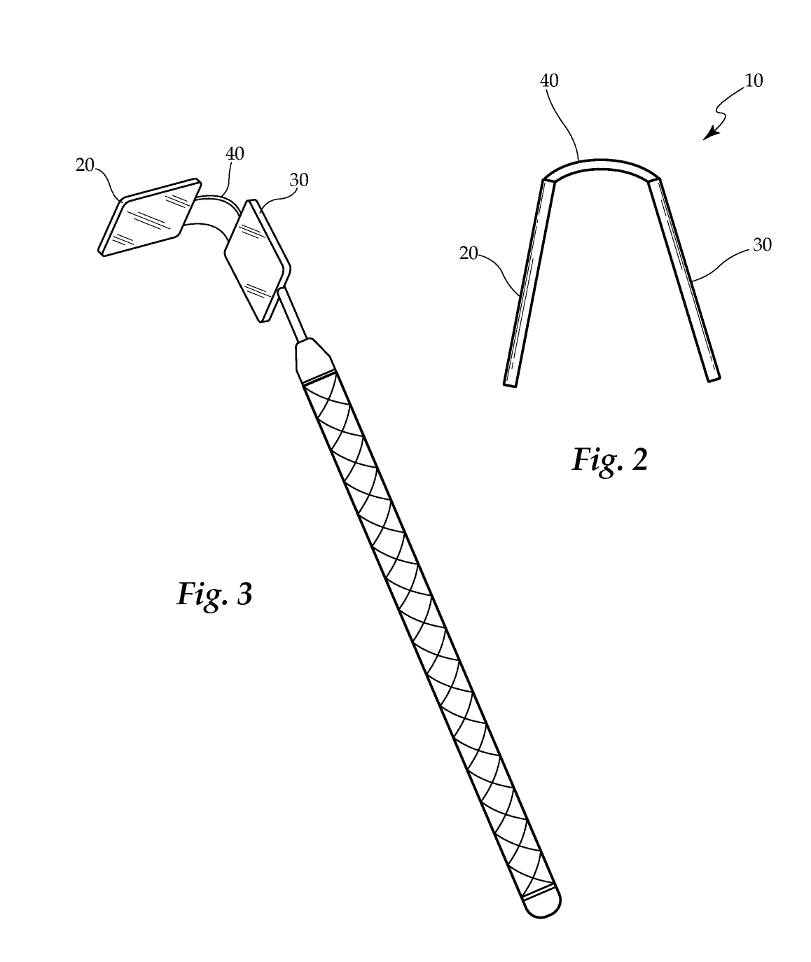

In an additional embodiment of the present system, a top view of the attachable head 10 is illustrated in FIG. 2. This view more clearly demonstrates the overall shape of the present design and its solid body form. The present invention is a unibody structure and can be composed of solid material without the need for numerous parts and attachments. The two rectangular retractors 20 30 have mirrored surfaces that oppose one another and form a lateral view for the dental practitioner, joined by bridge 40. This embodiment also emphasizes the size of the device and its capability of being place in one quadrant of the oral cavity without need for repositioning due to its sturdy structure and a substantially “V” or “U” shape 10.

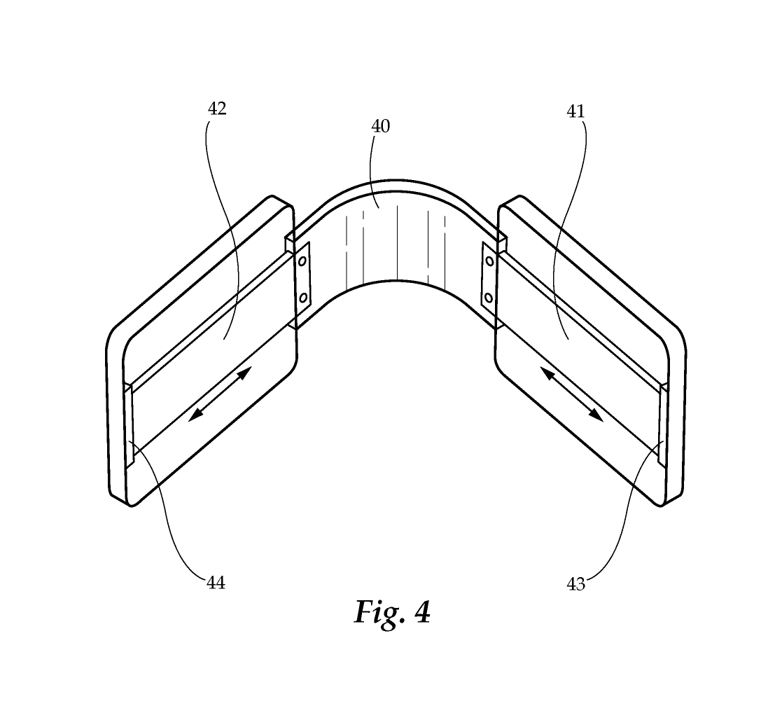

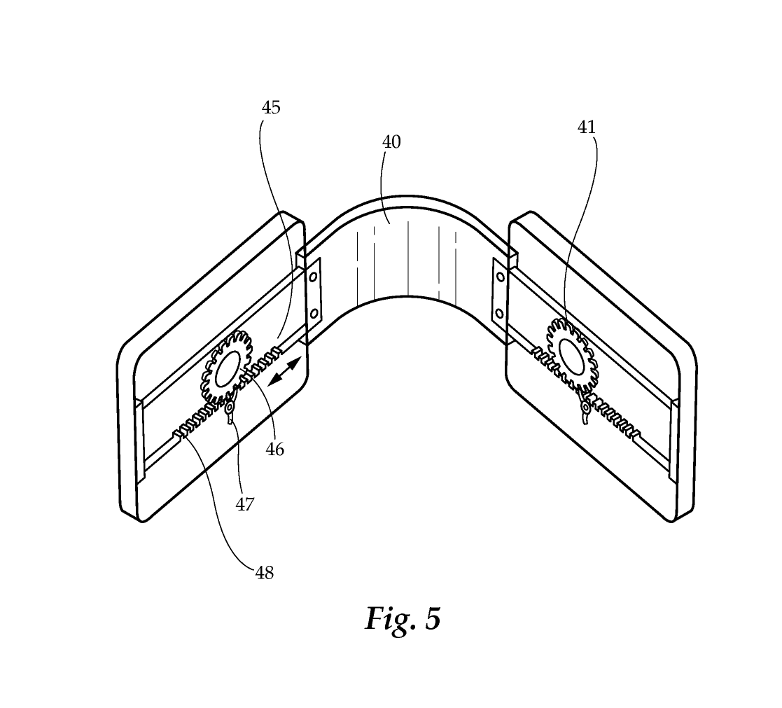

In one embodiment, the soft tissue retractor comprises a housing for the two mirrors wherein the mirrors are structured in a substantially linear alignment and are disposed to retain the mirror flat against the rectangular component. In a further embodiment of the present system is illustrated in FIG. 3 and emphasizes the bridge 40. The preferable design for the bridge connecting the two rectangular retractors 20, 30 is one with a minimal curve and forming an arch that is capable of resting behind the posterior molars and maintaining the overall shape of the device so as to retract tissue. In an additional embodiment, the bridge is capable of extension via a telescoping mechanism or a ratcheting mechanism as exemplified by FIG. 4 and FIG. 5. The telescoping mechanism can have an internal arm 41, 42 inside in the hollow interior of the rectangular retractor sides 20, 30. This interior arm is smaller than the hollowed bridge receiver and can slide in and out of the bridge from both sides. The present design features a stop 43, 44 on the end of the retractable arm that prevent the bridge piece from following out of the rectangular retractor sides. The interior arms can be manufactured in either the composite material mentioned above of the overall design or the metal. The present invention also considers a ratcheting mechanism as shown in FIG. 5 that could aid in adjustment of the bridge. The internal arm 45 can be made similar to the arm of the telescoping design but in this embodiment features notches or stops 48 that allows the pawl 47 to lock the arm into place and allow a ratcheting motion. The ratchet 46 incrementally extends the arm by pulling on the bridge 40.

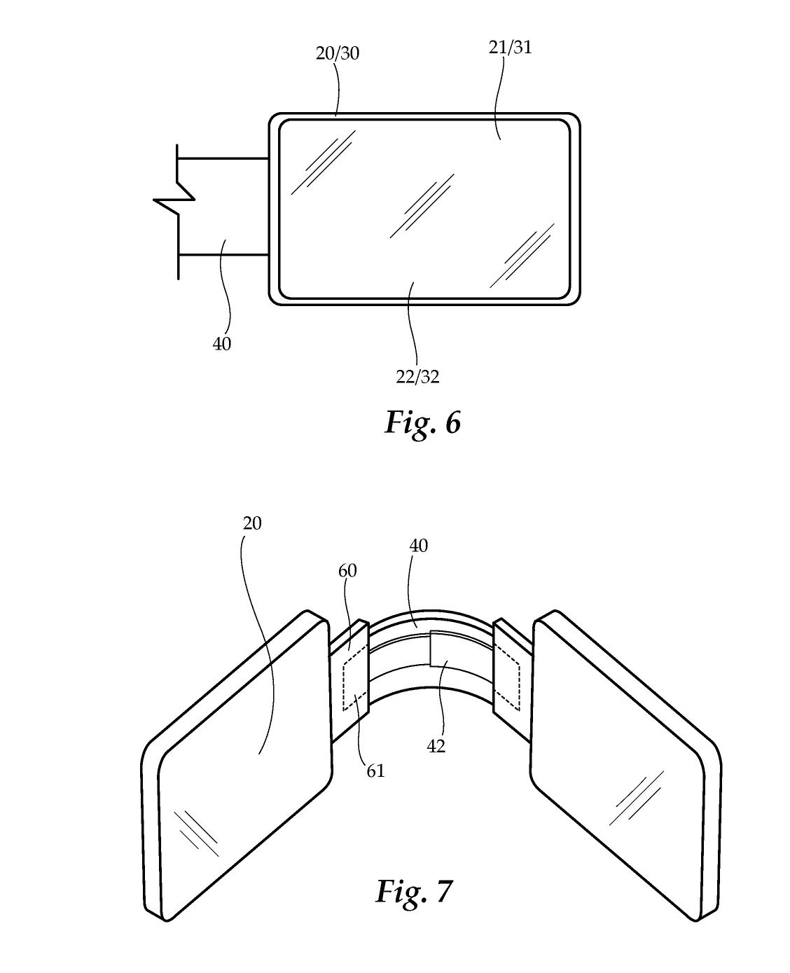

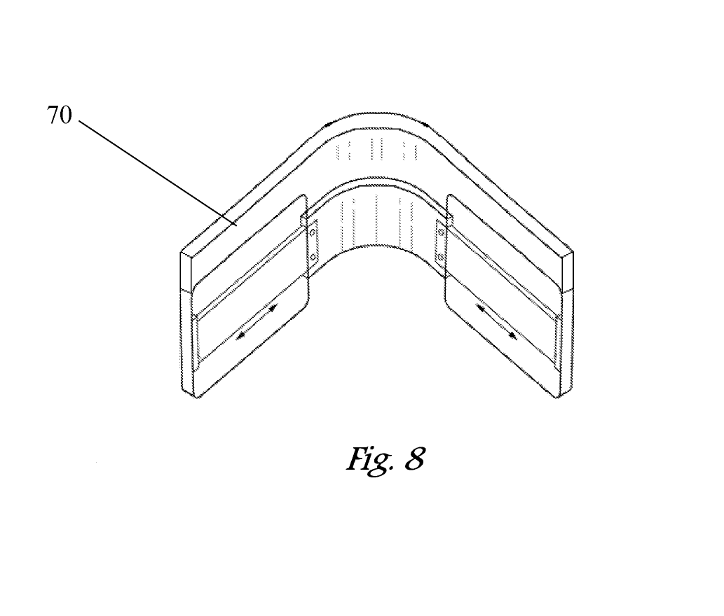

In an additional embodiment of this design is illustrated by FIG. 6. The rectangular retractor sides 20, 30 have attached mirrors 21, 31 that are adhered to the inside surface of each rectangle facing each other. The retractor sides are rectangular in shape but also feature rounded corners 22, 32. These corners aid in holding the mirrors in the housing of the rectangle. The mirror can be adhered by the use of glue, epoxy or any other adhesive that is safe for use in a human oral cavity on the back surface of the mirror and then is secured by clipping it into the housing 20, 30. Due to the linear and rectangular shape of the retractor sides the mirror is positioned flat against the linear plan and contains no convex or concave features as seen in the side profile crosscut view of the mirror in FIG. 6. The mirror 21, 31 is a reflective surface that when placed in opposition of another mirror yields a lateral view of the working area. The present invention allows for the mirror to be magnified allowing the practitioner a more precise view and in the case of adjustable bridge length it may house LED or other light source to improve vision and to allow the practitioner to complete the treatment in case of power failure. Additionally, FIG. 7 illustrates an isometric view of an additional embodiment of an adjustable mechanism of the bridge arm, illustrating supplementary sliding or retractable mechanism of the bridge arm and FIG. 8 illustrates an isometric view of an additional embodiment of an adjustable mechanism of within the bridge arm and a bite block 70 according to the present invention.

Moreover, in one embodiment, the instant soft tissue retractor may comprise a housing wherein the two mirrors are linear and thus the mirror will sit flat against a rectangular component. This linear edge provides stability and as such, when in position, reduces/eliminates the need for repositioning of the apparatus by the practitioner. Thus, the instant system further reduces or even eliminates the need for four handed dentistry and thus dental assistants can then be free to perform their other duties more efficiently.

Moreover the instant system may be utilized in conjunction with a bite guard or bite block apparatus which may be positioned above the bridge area. The patient would remain open while retraction is maintained. The instant invention could simply comprise a rubber bite block with a slot for the bridge to fit in. The bite guard or bite block may be composed of a suitable material for contact with a human oral surfaces including, appropriate metals, rubber, polymers, plastic, composites including resin composites and carbon fiber composites.

What I claim is:

1. A soft tissue retractor disposed to simultaneously retract and provide a clear working area, comprising:
at least two substantially reflective surfaces:
a unibody attachment head comprising:
at least two substantially rectangular components; and,
a bridge member comprising a radial orientation wherein the two rectangular retractors;
wherein the rectangular components are disposed to be large enough to fill the length of an entire quadrant of the oral cavity.

2. The soft tissue retractor disposed to simultaneously retract and provide a clear working area of claim 1 wherein at least one of the at least two substantially rectangular components comprises a socket area comprising a threaded receiving mechanism for attachment to any standard dental instrument handle.

3. The soft tissue retractor disposed to simultaneously retract and provide a clear working area of claim 1 wherein at least two substantially reflective surfaces comprise at least two mirrors affixed to the inside surface of at least two substantially rectangular components.

4. The soft tissue retractor disposed to simultaneously retract and provide a clear working area of claim 1 wherein the bridge comprises a telescoping extension mechanism.

5. The soft tissue retractor disposed to simultaneously retract and provide a clear working area of claim 1 wherein the bridge comprises an extension mechanism.

6. The soft tissue retractor disposed to simultaneously retract and provide a clear working area of claim 5 wherein the extension mechanism comprises a telescoping extension mechanism.

7. The soft tissue retractor disposed to simultaneously retract and provide a clear working area of claim 5 wherein the extension mechanism comprises a ratcheting mechanism.

8. The soft tissue retractor disposed to simultaneously retract and provide a clear working area of claim 1 wherein the angle between the at least two substantially reflective surfaces comprises an acute angle.

9. The soft tissue retractor disposed to simultaneously retract and provide a clear working area of claim 3, wherein the at least two substantially reflective surfaces of the inside of the rectangles can be magnified.

10. The soft tissue retractor disposed to simultaneously retract and provide a clear working area of claim 3 wherein an angle between the at least two mirrors comprises an acute angle.

11. The soft tissue retractor disposed to simultaneously retract and provide a clear working area of claim 3, wherein the at least two mirrors can be magnified.

12. A soft tissue retractor disposed to simultaneously retract and provide a clear working area of claim 3, wherein a housing of the at least two mirrors are linear and disposed to retain the mirror flat against the rectangular component.

13. The dental tool head attachment of claim 14 wherein the retractable bridge member comprises a bite block mechanism.

14. A dental tool head attachment comprising:
at a first substantially rectangular surface comprising radial corner sections and a second substantially rectangular surface comprising radial corner sections;
a retractable bridge member; and,
a substantially cylindrical threaded retaining mechanism; wherein the first substantially rectangular surface comprising radial corner sections and the second substantially rectangular surface comprising radial corner sections are in dynamic communication via the retractable bridge member.

15. The dental tool head attachment of claim 14 wherein the retractable bridge member comprises a telescoping extension mechanism.

16. The dental tool head attachment of claim 14 wherein the retractable bridge member comprises a ratcheting extension mechanism.

17. The dental tool head attachment of claim 14 wherein the retractable bridge member comprises a bite block mechanism.
